(19) United States
(12) Patent Application Publication
(10) Pub. No.: US 2025/0303197 A1
(43) Pub. Date: Oct. 2, 2025

(54) Fluid-Collection Ring for Thermal Therapy Device

(71) Applicant: Profound Medical Inc., Mississauga (CA)

(72) Inventors: Samantha Lee, Mississauga (CA); Yifan Yang, Maple (CA); Hooman Reza Zadeh Tabatabai, Richmond Hill (CA); Christopher Driver, Oakville (CA)

(21) Appl. No.: 19/062,391

(22) Filed: Feb. 25, 2025

Related U.S. Application Data
(60) Provisional application No. 63/573,055, filed on Apr. 2, 2024.

(51) Int. Cl. A61N 7/00; A61B 90/00

(52) U.S. Cl. CPC A61N 7/00; A61B 2090/0472; A61N 2007/0043

(57) ABSTRACT

A medical device comprises an ultrasound applicator comprising a shaft having proximal and distal ends; a plurality of channels defined in the shaft and extending from the proximal end of the shaft, including an ultrasound channel and a cooling channel; one or more ultrasound transducers disposed in the ultrasound channel; an ultrasound window defined in the shaft and aligned with the one or more ultrasound transducers; and a handle attached to the proximal end of the shaft. A fluid-collection ring is disposed on a distal side of the handle. The fluid-collection ring has an inner edge and an outer edge, the inner edge defining a hole through which the shaft extends. A distal side of the fluid-collection ring has a concave shape and is configured to direct fluid away from the handle.

### CROSS-REFERENCE TO RELATED APPLICATIONS

This application claims priority to U.S. Provisional Application No. 63/573,055, titled “Fluid-Collection Ring for Thermal Therapy Device,” filed on Apr. 2, 2024, which is hereby incorporated by reference.

### TECHNICAL FIELD

This application relates generally to ultrasound therapy devices.

### BACKGROUND

Ultrasound therapy devices are used to treat a variety of conditions including benign prostatic hyperplasia (BHP), calcifications such as bladder stones and kidney stones, and tumors. For some conditions, an ultrasound probe is inserted into the body to place the ultrasound transducers close to the target region. During a medical procedure, bodily fluids and/or fluids injected into the body opening can flow from the patient onto the inserted ultrasound probe and/or other equipment supporting the device such as a positioning system which may cause damage thereto.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a medical device comprising an ultrasound applicator comprising a shaft having proximal and distal ends; a plurality of channels defined in the shaft and extending from the proximal end of the shaft, including: an ultrasound channel, and a cooling channel; one or more ultrasound transducers disposed in the ultrasound channel; an ultrasound window defined in the shaft and aligned with the one or more ultrasound transducers; and a handle attached to the proximal end of the shaft. The medical device further comprises a fluid-collection ring disposed on a distal side of the handle, the fluid-collection ring having an inner edge and an outer edge, the inner edge defining a hole through which the shaft extends, a distal side of the fluid-collection ring having a concave shape and configured to direct fluid away from the handle.

In one or more embodiments, the distal side of the fluid-collection ring is configured to conform to an entry point of a mammalian body. In one or more embodiments, the distal side of the fluid-collection ring is configured to conform to a tip of a penis. In one or more embodiments, a proximal side of the fluid-collection ring is attached to the distal side of the handle such that a rotational orientation of the fluid-collection ring remains stationary when the shaft is rotated.

In one or more embodiments, the inner edge and the shaft form a fluid-tight seal. In one or more embodiments, the inner edge and the shaft define a fluid-collection channel and the medical device further comprises a fluid-collection reservoir that is fluidly coupled to the fluid-collection channel. In one or more embodiments, the fluid-collection reservoir is disposed in the handle.

In one or more embodiments, the medical device further comprises complementary keyed projections disposed on a proximal side of the fluid-collection ring and the distal side of the handle, the complementary keyed projections configured to mechanically engage one another to limit a rotation of the fluid-collection ring with respect to the shaft.

In one or more embodiments, wherein the fluid-collection ring comprises a biocompatible polymer or a biocompatible plastic. In one or more embodiments, a drainage hole is defined through a proximal side and the distal side of the fluid-collection ring and the medical device further comprises a drainage tube fluidly coupled to the drainage hole on the proximal side of the fluid-collection ring. In one or more embodiments, the fluid-collection ring is elongated with respect to a first axis compared to with respect to a second axis, the shaft extending along a third axis, the first, second, and third axes mutually orthogonal.

Another aspect of the invention is directed to a medical device comprising an ultrasound applicator comprising a shaft having proximal and distal ends; a plurality of channels defined in the shaft and extending from the proximal end of the shaft, including: an ultrasound channel, and a cooling channel; one or more ultrasound transducers disposed in the ultrasound channel; an ultrasound window defined in the shaft and aligned with the one or more ultrasound transducers; and a handle attached to the proximal end of the shaft. The medical device further comprises a fluid-collection ring disposed on a distal side of the handle, the fluid-collection ring having an inner edge, an outer edge, and a fluid-collection channel defined on a distal side of the fluid-collection ring.

In one or more embodiments, the fluid-collection channel is defined only on a distal surface of the fluid-collection ring. In one or more embodiments, the fluid-collection channel extends to a hole defined in the outer edge of the fluid-collection ring. In one or more embodiments, the hole is oriented at a bottom of the fluid-collection channel when the shaft is oriented parallel to a horizontal axis.

Another aspect of the invention is directed to a medical apparatus comprising an ultrasound applicator comprising a shaft having proximal and distal ends; a plurality of channels defined in the shaft and extending from the proximal end of the shaft, including: an ultrasound channel, and a cooling channel; one or more ultrasound transducers disposed in the ultrasound channel; an ultrasound window defined in the shaft and aligned with the one or more ultrasound transducers; and a handle attached to the proximal end of the shaft. The medical apparatus further comprises a fluid-collection ring disposed on a distal side of the handle, the fluid-collection ring having an inner edge and an outer edge, the inner edge defining a hole through which the shaft extends, a distal side of the fluid-collection ring having a concave shape and configured to direct fluid away from the handle; a power supply electrically coupled to the one or more ultrasound transducers; a reservoir configured to store a cooling fluid, the reservoir fluidly coupled to the cooling channel; and a pump that circulates the cooling fluid between the reservoir and the cooling channel.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a block diagram of a medical system according to one or more embodiments.

FIG. 2 is a simplified and partially transparent side view of a medical apparatus according to one or more embodiments.

FIG. 3A is a simplified and partially transparent side view of a medical apparatus according to one or more embodiments.

FIGS. 3B and 3C are cross sections of the medical apparatus illustrated in FIG. 3A according to one or more embodiments.

FIG. 4 is a simplified and partially transparent side view of a medical apparatus according to one or more embodiments.

FIG. 5A is an isometric view of an ultrasound applicator according to one or more embodiments.

FIG. 5B is an enlarged view of a portion of the ultrasound applicator illustrated in FIG. 5A according to one or more embodiments.

FIGS. 6A and 6B are isolated isometric views of the fluid-collection ring, shown in FIGS. 5A and 5B, from different perspectives.

FIG. 7A is an enlarged view of a portion of the ultrasound applicator and a fluid-collection ring according to one or more embodiments.

FIG. 7B is an isolated isometric view of the fluid-collection ring shown in FIG. 7A according to one or more embodiments.

FIG. 8A is an enlarged view of a portion of the ultrasound applicator and a fluid-collection ring according to one or more embodiments.

FIG. 8B is an isolated isometric view of the fluid-collection ring shown in FIG. 8A according to one or more embodiments.

FIG. 9A is an enlarged view of a portion of the ultrasound applicator and a fluid-collection ring according to one or more embodiments.

FIG. 9B is an isolated isometric view of the fluid-collection ring shown in FIG. 9A according to one or more embodiments.

FIG. 10A is an enlarged view of a portion of the ultrasound applicator and a fluid-collection ring according to one or more embodiments.

FIG. 10B is an isolated isometric view of the fluid-collection ring shown in FIG. 10A according to one or more embodiments.

FIG. 11A is an enlarged view of a portion of the ultrasound applicator and a fluid-collection ring according to one or more embodiments.

FIG. 11B is an isolated isometric view of the fluid-collection ring shown in FIG. 11A according to one or more embodiments.

### DETAILED DESCRIPTION

A medical device includes an ultrasound applicator and a fluid-collection ring. The ultrasound applicator includes a shaft and a handle attached to the shaft. The fluid-collection ring is disposed on a distal end of the handle and has a hole through which the shaft extends. The fluid-collection ring is configured to collect and/or deflect liquids, such as bodily fluids, that may discharge from a patient during a medical procedure. The fluid-collection ring can alternately be referred to as a fluid-deflection ring. The ultrasound applicator can be coupled to a cooling-fluid reservoir, a power source, and/or a controller to form a medical apparatus.

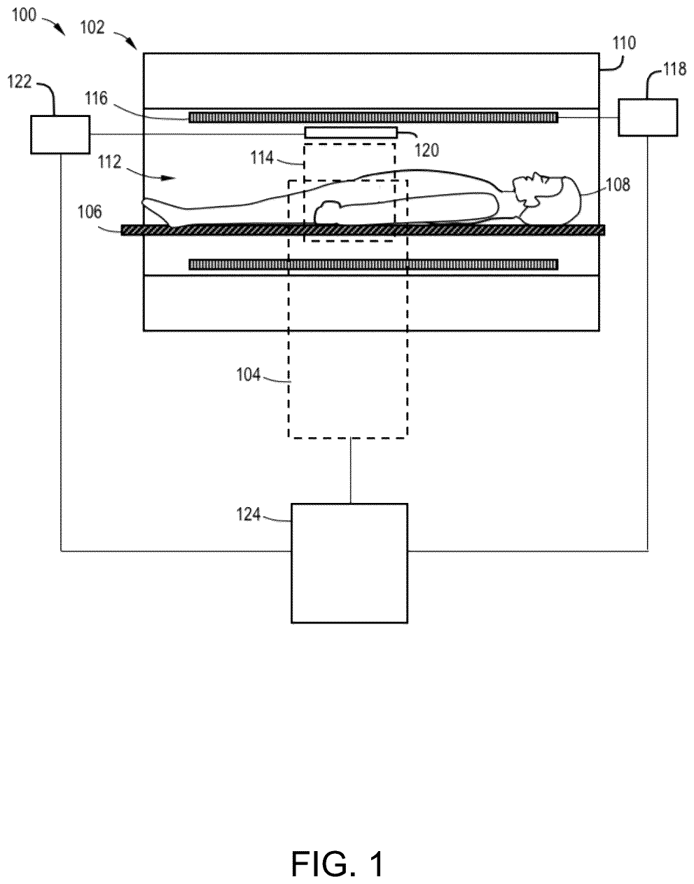

FIG. 1 is a block diagram of a medical system 100 in which at least some of the apparatus, systems, and/or methods disclosed herein are employed, in accordance with at least some embodiments. The system 100 includes a patient support 106 (on which a patient 108 is shown), a magnetic resonance imaging (MRI) system 102 or other imaging system is utilized as well as an image-guided energy delivery system 104.

The magnetic resonance system 102 includes a magnet 110 disposed about an opening 112, an imaging zone 114 in which the magnetic field is strong and uniform enough to perform MRI, a set of magnetic field gradient coils 116 to change the magnetic field rapidly to enable the spatial coding of MRI signals, a magnetic field gradient coil power supply 118 that supplies current to the magnetic field gradient coils 116 and is controlled as a function of time, a transmit/receive coil 120 (also known as a “body” coil) to manipulate the orientations of magnetic spins within the imaging zone 114, a radio frequency transceiver 122 connected to the transmit/receive coil 120, and a computer 124, which performs tasks (by executing instructions and/or otherwise) to facilitate operation of the MRI system 102 and is coupled to the radio frequency transceiver 122, the magnetic field gradient coil power supply 118, and the image-guided energy delivery system 104. The image-guided energy delivery system 104 includes a therapeutic applicator, such as an ultrasound applicator, to perform image-guided therapy (e.g., thermal therapy) to treat a treatment volume in the patient 108.

The MRI computer 124 can include more than one computer in some embodiments, at least one of which can be dedicated to the MRI system 102. In at least some embodiments, the MRI computer 124 and/or one or more other computing devices (not shown) in and/or coupled to the system 100 may also perform one or more tasks (by executing instructions and/or otherwise) such as to control the driving or operating frequency of the ultrasound elements in the therapeutic applicator, such as at the center frequency (f₀) and/or at a higher harmonic (3f₀) of the center frequency.

One or more of the computers, including computer 124, can include a treatment plan for and/or program instructions for determining a treatment plan (e.g., in real time) for the patient 108 that includes the target treatment volume and the desired or minimal energy (e.g., thermal) dose for the target treatment volume. The treatment plan can also include the desired operating or driving frequency of the ultrasound elements, such as f₀ and/or 3f₀. The computer(s) can use images from the MRI or other imaging system 102 to image guide the rotational position and insertion-retraction position of the therapeutic applicator. In some embodiments, one or more dedicated computers control the image-guided energy delivery system 104. Some or all of the foregoing computers can be in communication with one another (e.g., over a local area network, a wide area network, a cellular network, a WiFi network, or other network), for example through a software-controlled link to a communication network.

In some embodiments, the treatment plan includes a set of initial parameters for driving each ultrasound element such as its initial frequency, initial phase, and initial amplitude. These parameters can be updated in real time based on the measured temperature of the target volume, for example as determined by MR thermometry.

The magnetic resonance system 102 can be replaced with another imaging system such as an ultrasound imaging system. Alternatively, the image-guided energy delivery system 104 can be used without an imaging system in which case the image-guided energy delivery system 104 can be referred to as an energy delivery system 104.

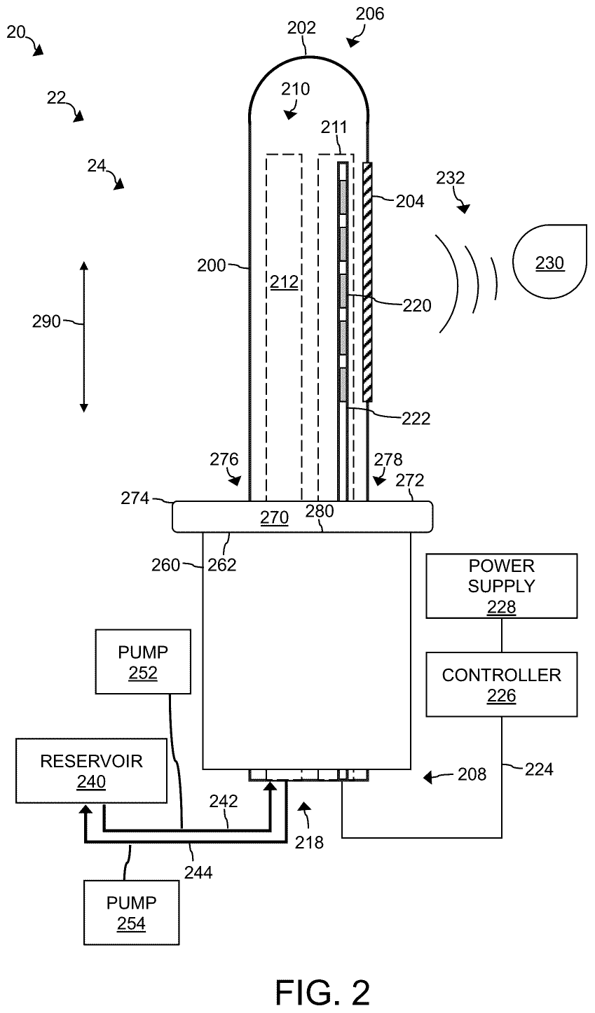

FIG. 2 is a simplified and partially transparent side view of a medical apparatus 20 according to one or more embodiments. The medical apparatus 20 includes a medical device 22 that includes an ultrasound applicator 24 and a fluid-collection ring 270. The ultrasound applicator 24 can be a therapeutic applicator for an image-guided energy delivery system 104 (or an energy delivery system 104) (FIG. 1). The medical apparatus 20 also includes a cooling fluid reservoir 240, an optional pump 252, 254, a controller 226, and a power supply 228.

The ultrasound applicator 24 includes a shaft 200 attached to or including a tip 202 at a distal end 206 of the ultrasound applicator 24. Multiple channels 210 can be defined in the shaft 200. Each channel 210 extends from a proximal end 218 of the shaft 200 parallel to an axis 290. The channels 210 can include an ultrasound channel 211 and/or a cooling channel 212.

The ultrasound channel 211 can be configured to receive one or more ultrasound transducers 220. The ultrasound transducer(s) 220 can comprise an array of ultrasound transducers (e.g., ultrasound transducer elements), such as a linear array or a focused array of ultrasound transducers. Alternatively, the ultrasound transducer(s) 220 can comprise a single ultrasound transducer or multiple ultrasound transducers that may not be arranged in an array. The ultrasound transducer(s) 220 can be mounted on and/or electrically connected to an elongated circuit board 222. The elongated circuit board 222 can be electrically coupled (e.g., via wire(s) 224) to the controller 226 that can selectively provide electrical power, produced by the power supply 228, at a frequency, relative phase, and/or amplitude according to a treatment plan so as to treat a target volume 230 in a mammalian patient. The controller 226 and the power supply 228 can be combined in some embodiments. Ultrasound energy 232 produced by the ultrasound transducer(s) 220 can pass through an ultrasound window 204 formed or defined in the shaft 200 and can be focused, geometrically and/or electronically (or otherwise), onto the target volume 230.

The cooling channel 212 can be configured to receive cooling fluid that can be used to cool the ultrasound applicator 24 and/or the surrounding volume (e.g., surrounding tissue) during ultrasonic treatment. The cooling fluid can be provided from a cooling fluid reservoir 240, such as an intravenous (IV) fluid bag. The cooling fluid can be recirculated between the cooling fluid reservoir 240 and the cooling channel 212. Cooler (e.g., room temperature) cooling fluid can flow from the cooling fluid reservoir 240 to the cooling channel 212 through an inlet line 242. After passing through at least a portion of the cooling channel 212 and receiving heat from the ultrasound applicator 24, warmer cooling fluid can flow from cooling channel 212 to the cooling fluid reservoir 240 through an outlet line 244. A pump 252 can be fluidly coupled to the inlet line 242 and/or a pump 254 can be fluidly coupled to the outlet line 244. Alternatively, pump 252 or 254 can be fluidly coupled to the inlet line 242 and to the outlet line 244.

A handle 260 is disposed on a proximal end 208 of the ultrasound applicator 24 and/or on the proximal end 218 of the shaft 200. The handle 260 can have a larger diameter or width than the shaft 200. The handle 260 is configured to be held by a provider or by a machine, such as an automated positioning system, during treatment. The handle 260 is configured to remain outside of the body while the shaft 200 is configured to be inserted into the body, such as into a natural or artificial (e.g., surgical) opening in the body. The handle 260 is shown as slightly offset from the proximal end 208 to show the proximal end of the shaft 200 and the ultrasound and cooling channels 211, 212 for illustration purposes only. The handle 260 can cover the proximal end of the shaft 200 in one or more embodiments.

The fluid-collection ring 270 is disposed on a distal side 262 of the handle 260. The fluid-collection ring 270 has an inner edge 276 that defines a hole 278 that is configured to receive the shaft 200.

The fluid-collection ring 270 can be configured to receive and/or collect fluids (e.g., liquids) during use of the ultrasound applicator 24. Additionally or alternatively, the fluid-collection ring 270 can be configured to deflect fluids away from the handle 260 and/or other equipment such as the power supply 228, the controller 226, the pump(s) 252, 254, and/or a positioning system (e.g., an automated positioning system).

A distal side 272 of the fluid-collection ring 270 can includes a taper, angle, or curve such that, in the orientation illustrated, an outer edge 274 is higher than an inner edge 276. In some embodiments, the distal side 272 has a concave shape, for example that can be formed by a portion of a sphere. The difference in height between the outer edge 274 and the inner edge 276 can cause fluids to flow towards the inner edge 276 and/or away from the handle 260 and other components of the medical apparatus 20 such as the power supply 228, the controller 226, and/or the pump(s) 252, 254.

During a medical procedure, the shaft 200 may be inserted into a natural opening or a surgical opening in a mammalian patient. For example, the shaft 200 may be inserted suprapubicly, transversally, transrectally, transperineally, or transurethrally. When the shaft 200 is inserted into a natural opening such as the urethra, the distal side 272 of the fluid-collection ring 270 may physically contact the head of a patient's penis. The distal side 272 can conform to the head of a penis to improve patient comfort and/or to better collect any fluids discharged (e.g., from the male urethra) during the medical procedure. Additionally or alternatively, the distal side 272 can conform to another anatomical feature at or near the natural opening or the surgical opening.

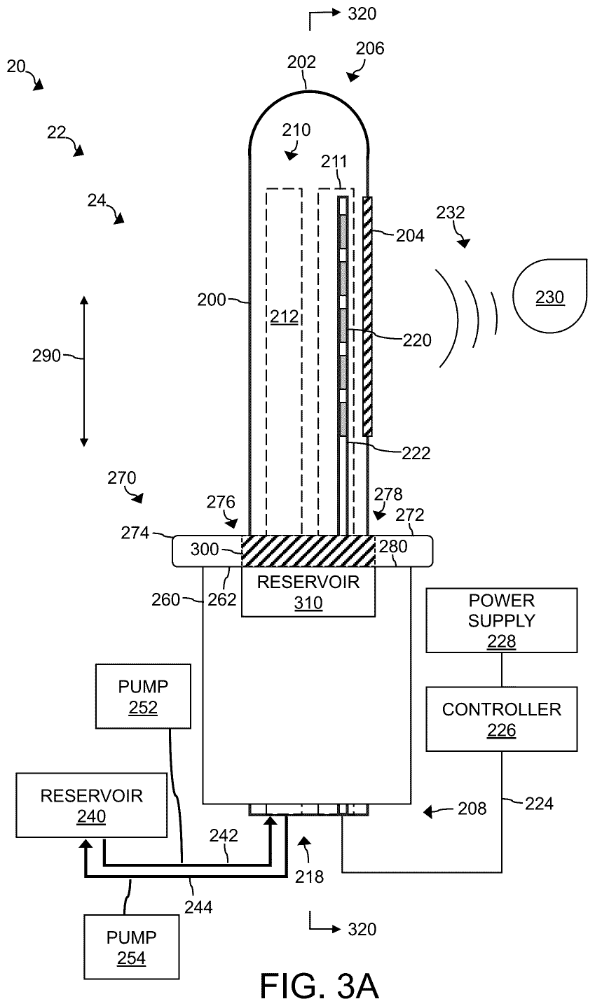

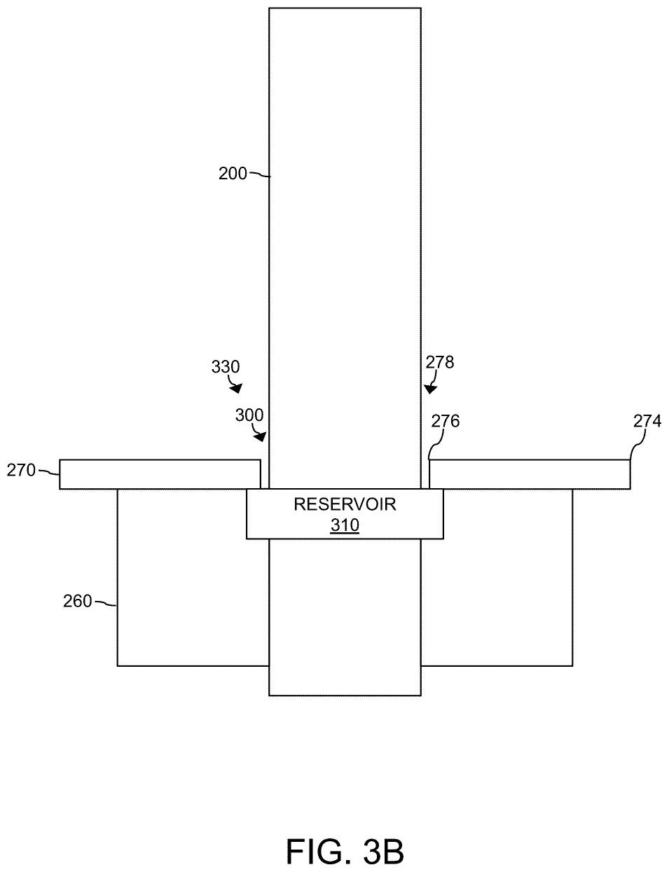

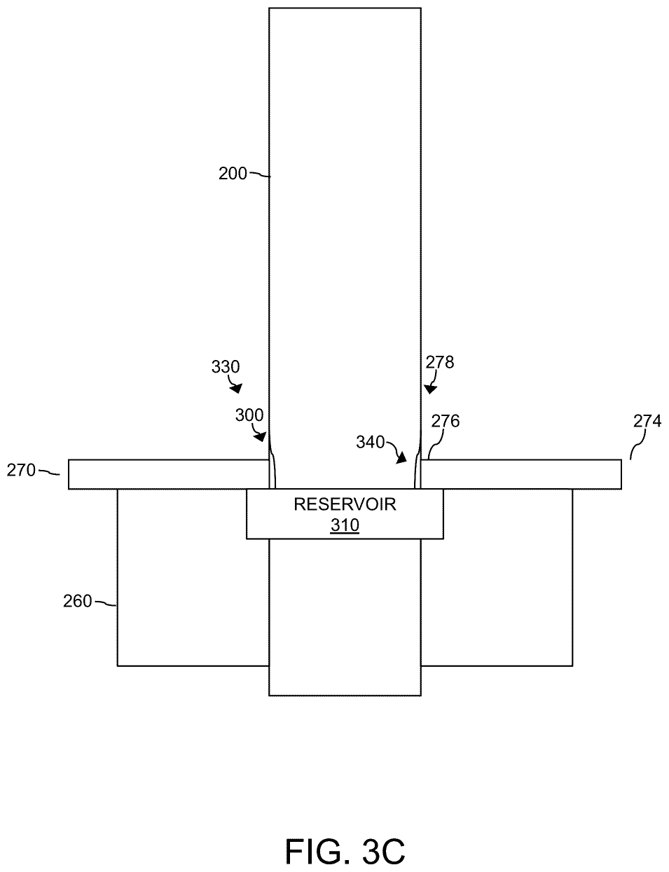

In an embodiment, the inner edge 276 and the shaft 200 form a fluid-tight seal. Any fluid discharged during a medical procedure can be collected and held on the distal side 272 of the fluid-collection ring 270 (e.g., depending on the orientation of shaft). In one or more embodiments, a channel 300 is defined between the inner edge 276 and the shaft 200, the channel 300 extending along the outside (e.g., perimeter or circumference) of the shaft 200, as illustrated in FIG. 3A. Any fluid discharged during a medical procedure can be collected on the distal side 272 of the fluid-collection ring 270 and funneled or directed into the channel 300. The channel 300 can be fluidly connected to a fluid-collection reservoir 310 in the handle 260. Collecting the discharged fluids prevents them from flowing onto and/or into nearby equipment, such as pump(s) 252, 254, a positioning system (e.g., an automated positioning system), or other equipment, and from flowing onto the floor. The channel 300 can be formed and/or defined by a gap 330 between the inner edge 276 of the fluid-collection ring 270 and the shaft 200, as shown in FIG. 3B which is a cross section of the ultrasound applicator 24 taken through plane 320 in FIG. 3A according to one or more embodiments. Additionally or alternatively, the channel 300 and/or the gap 330 can be formed and/or defined by a groove 340 in the shaft 200 adjacent to the inner edge 276 of the fluid-collection ring 270, as shown in FIG. 3C which is a cross section of the ultrasound applicator 24 taken through plane 320 in FIG. 3A according to one or more embodiments.

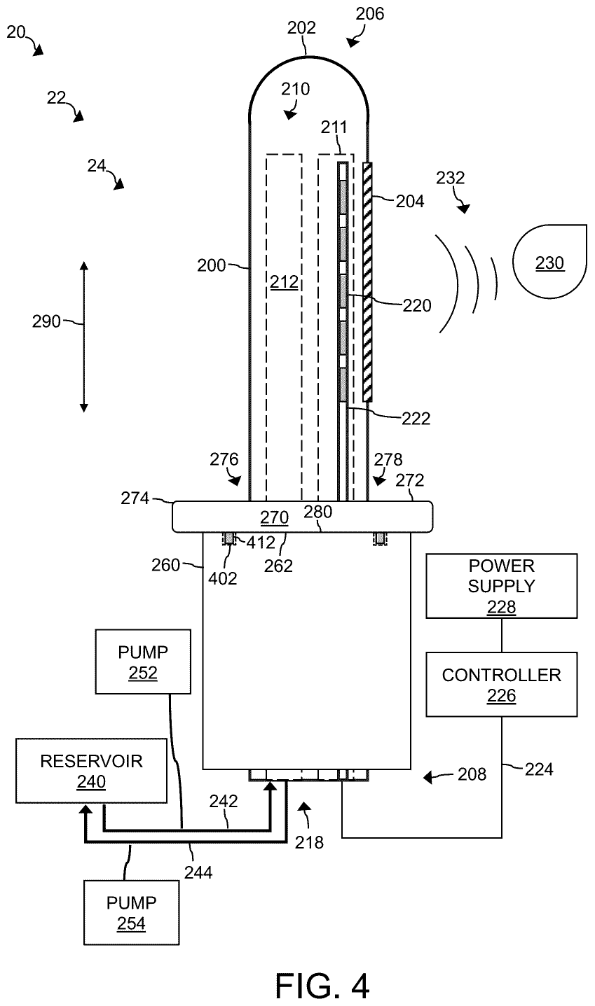

In one or more embodiments, a proximal side 280 (FIGS. 2, 3A) of the fluid-collection ring 270 can be attached to the distal side 262 of the handle 260 such that the rotational orientation of the fluid-collection ring 270 remains stationary as the shaft 200 is rotated during therapy. Additionally or alternatively, the distal side 262 of the handle 260 can include one or more features 402 that mechanically engage one or more respective features 412 on the proximal side 280 of the fluid-collection ring 270, as illustrated in FIG. 4, for example keyed and/or complementary features on the distal side 262 of the handle 260 and on the proximal side 280 of the fluid-collection ring 270. The embodiment illustrated in FIG. 4 can be combined with the embodiments illustrated in FIGS. 2, 3A, 3B, and/or 3C.

The fluid-collection ring 270 can be removable in some embodiments. For example, depending on the size of the head of the patient's penis (or other anatomical feature to which the fluid-collection ring 270 can conform), the fluid-collection ring 270 may be removed and replaced with another fluid-collection ring that has a larger or smaller size that more closely matches the size of the head of the patient's penis (or other anatomical feature to which the fluid-collection ring 270 can conform).

The fluid-collection ring 270 can be formed out of a biocompatible material such as a biocompatible polymer or plastic. The fluid-collection ring 270 can be configured to be placed on other thermal therapy devices instead of the ultrasound applicator 24.

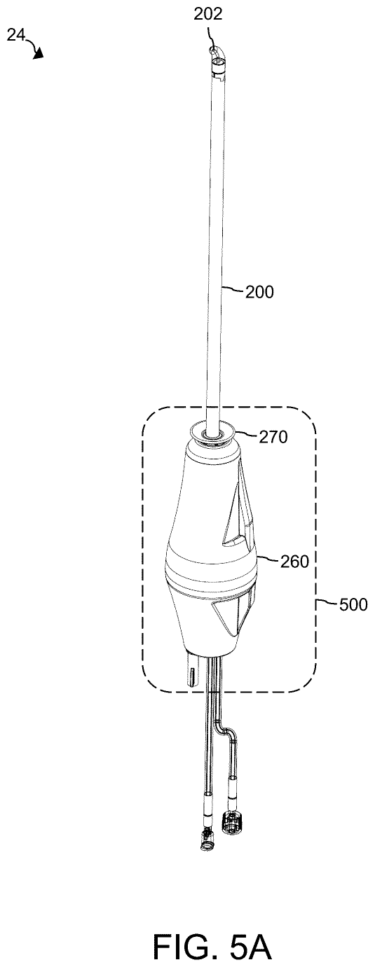

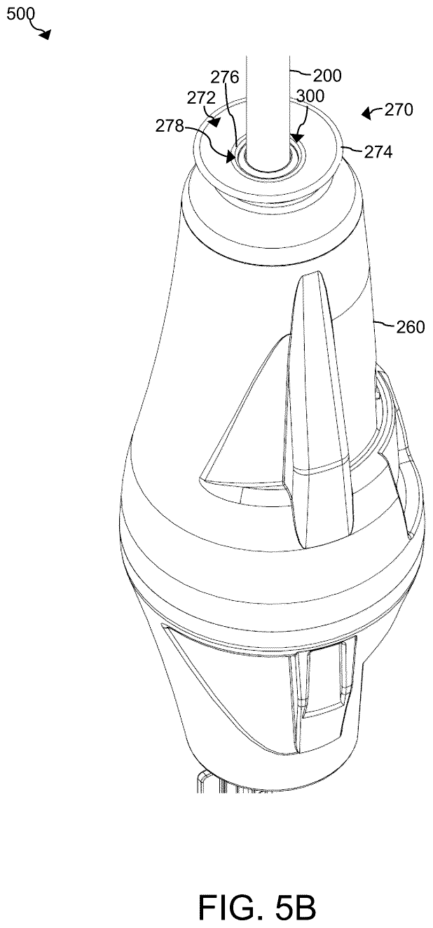

FIG. 5A is an isometric view of the ultrasound applicator 24 according to one or more embodiments to further illustrate the fluid-collection ring 270.

FIG. 5B is an enlarged view of region 500 in FIG. 5A to further illustrate the fluid-collection ring 270 including inner edge 276, the channel 300, and an example concave distal side 272. The distal side 272 of the fluid-collection ring 270 has a circular outer edge 274. In other embodiments, the outer edge 274 can define an oval, a hexagon, an octagon, or another shape.

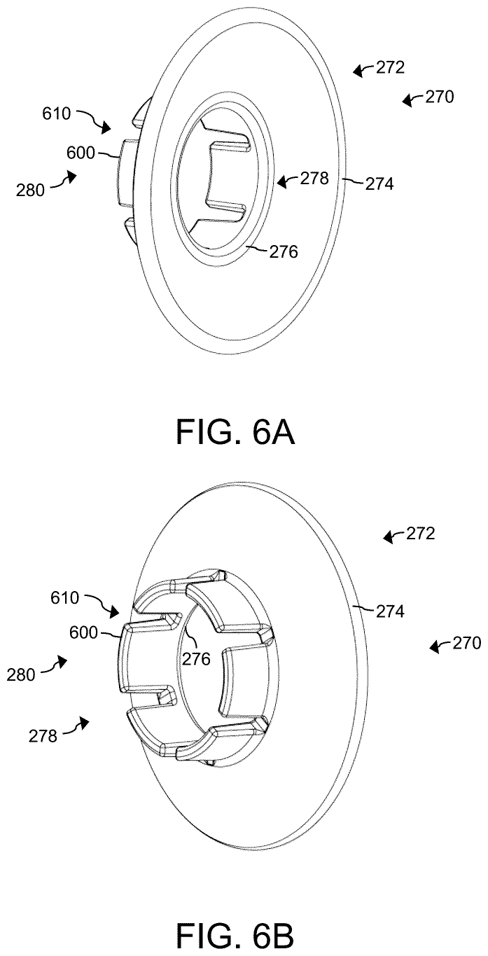

FIGS. 6A and 6B are isolated isometric views of the fluid-collection ring 270, shown in FIGS. 5A and 5B, from different perspectives to further illustrate the distal sides and proximal sides 272, 280, respectively. A plurality of tabs 600 are attached to the inner edge 276 of the fluid-collection ring 270. The tabs 600 are configured to mechanically engage the shaft 200 to mechanically secure the fluid-collection ring 270 to the shaft 200. In some embodiments, the handle 260 can include complementary tabs or ridges that are configured to mechanically engage the gaps 610 between neighboring tabs 600 for example to prevent rotation of the fluid-collection ring 270 relative to the handle 260 while allowing rotation of the fluid-collection ring 270 relative to the shaft 200.

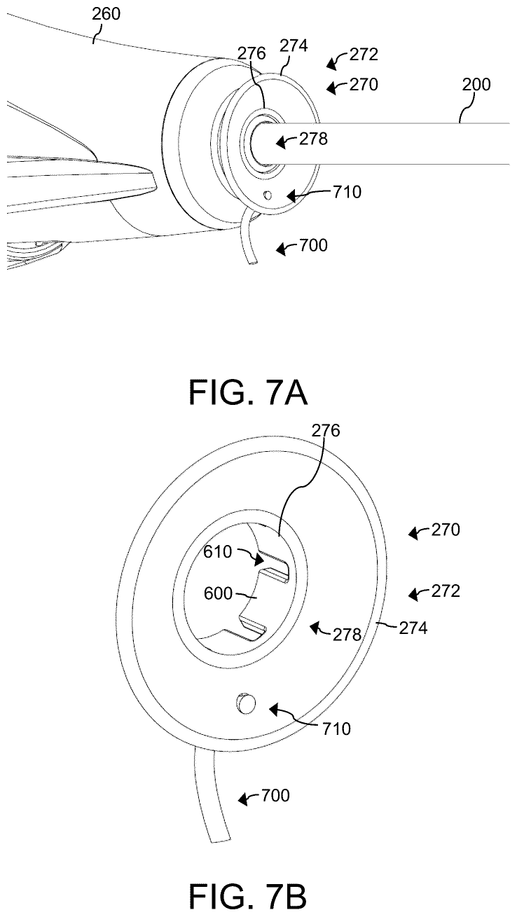

FIGS. 7A and 7B illustrate a fluid-collection ring 270 having a drainage tube 700 that is coupled to a fluid-drainage hole 710 in the fluid-collection ring 270. Other than the drainage tube 700 and the fluid-drainage hole 710, the fluid-collecting ring 270 illustrated in FIGS. 7A and 7B can be the same as the fluid-collection ring 270 shown in FIGS. 2, 3A, 3B, 3C, 4, 5A, 5B, 6A, and/or 6B. The fluid-collection ring 270 can be configured such that when the shaft 200 is oriented horizontally (or approximately horizontally such as within about 15 degrees of a horizontal plane), the fluid-drainage hole 710 is located below the shaft 200 so as to be in position to collect fluids that may flow downward on the distal side 272 of the fluid-collecting ring 270 during a medical procedure.

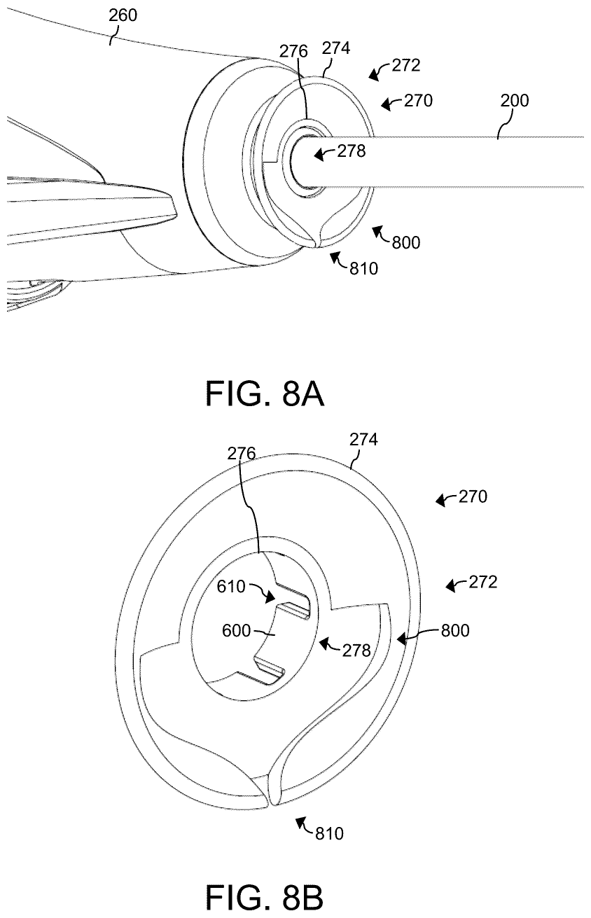

FIGS. 8A and 8B illustrate a fluid-collection ring 270 having a fluid-drainage channel or recess 800 defined in the distal side or surface 272 of the fluid-collection ring 270. The fluid-drainage channel or recess 800 can extend to a fluid-drainage hole 810 that can be defined in the outer edge 274 of the fluid-collection ring 270. The fluid-collection ring 270 can be configured such that when the shaft 200 is oriented horizontally (or approximately horizontally such as within about 15 degrees of a horizontal plane), the fluid-drainage channel or recess 800 and the fluid-drainage hole 810 are located below the shaft 200 so as to be in position to collect fluids that may flow downward on the distal side 272 of the fluid-collecting ring 270 during a medical procedure.

Other than the fluid-drainage channel or recess 800 and the fluid-drainage hole 810, the fluid-collecting ring 270 illustrated in FIGS. 8A and 8B can be the same as or combined with the fluid-collection ring 270 shown in FIGS. 2, 3A, 3B, 3C, 4, 5A, 5B, 6A, 6B, 7A, and/or 7B.

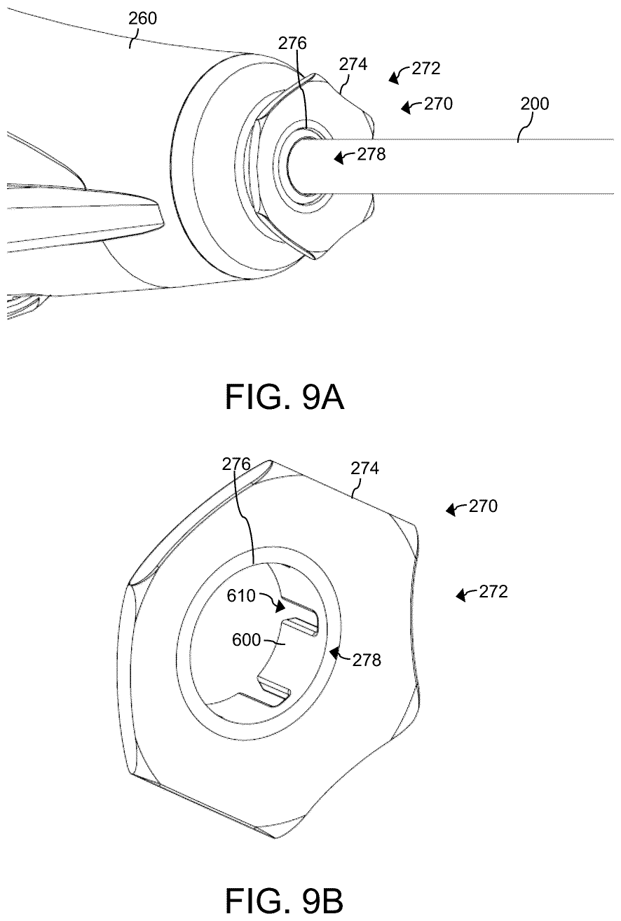

FIGS. 9A and 9B illustrate a fluid-collection ring 270 having an outer edge 274 that is hexagonal. The outer edge 274 can be another shape, such as another polygonal shape, in other embodiments.

Other than the shape of the outer edge 274, the fluid-collecting ring 270 illustrated in FIGS. 9A and 9B can be the same as or combined with the fluid-collection ring 270 shown in FIGS. 2, 3A, 3B, 3C, 4, 5A, 5B, 6A, 6B, 7A, 7B, 8A, and/or 8B.

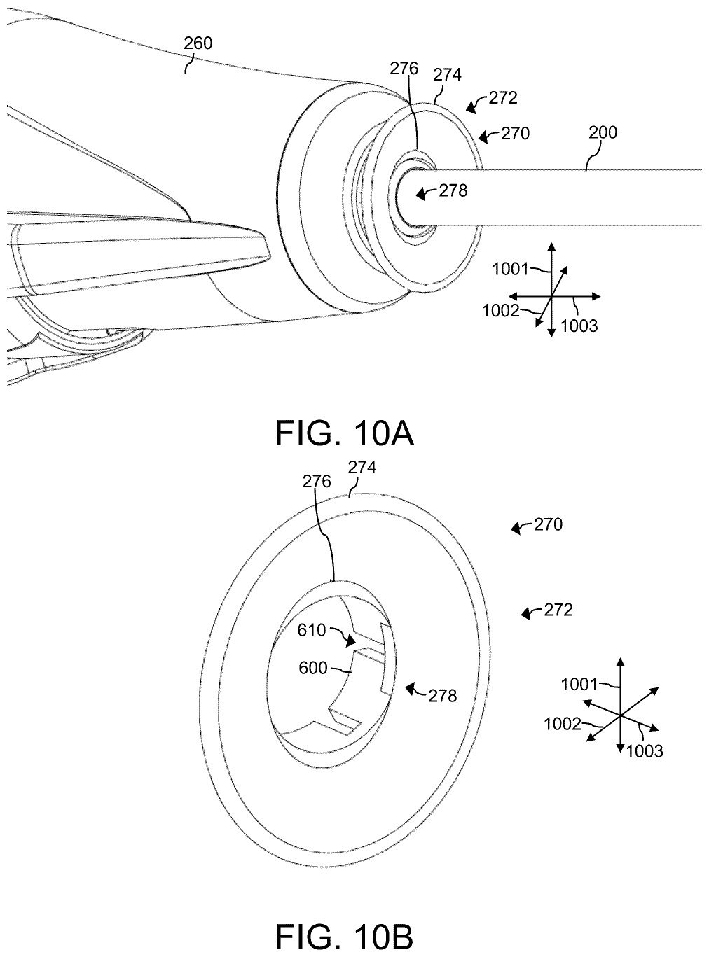

FIGS. 10A and 10B illustrate a fluid-collection ring 270 having an oval shape. The fluid-collection ring 270 is elongated with respect to a first axis 1001 compared to with respect to a second axis 1002 that is orthogonal to the first axis 1001. The shaft 200 extends along or parallel to a third axis 1003 that is orthogonal to the first and second axes 1001, 1002. Thus, axes 1001-1003 are mutually orthogonal.

Other than the shape of the fluid-collection ring 270, the fluid-collecting ring 270 illustrated in FIGS. 10A and 10B can be the same as or combined with the fluid-collection ring 270 shown in FIGS. 2, 3A, 3B, 3C, 4, 5A, 5B, 6A, 6B, 7A, 7B, 8A, 8B, 9A, and/or 9B.

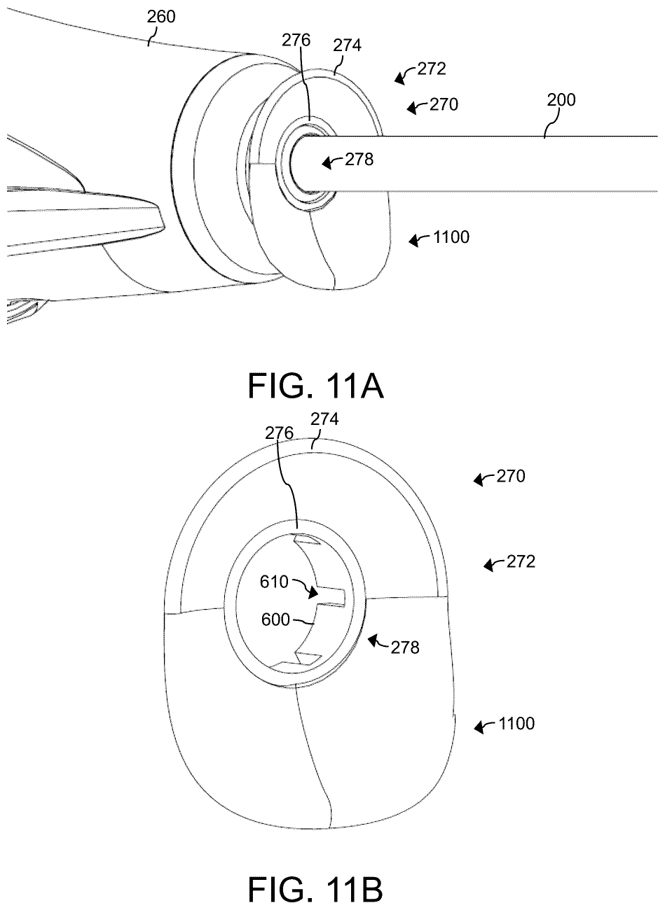

FIGS. 11A and 11B illustrate a fluid-collection ring 270 having an oval shape and a flared portion 1100. The flared portion 1100 can function as a channel that directs fluids downward and away from the handle 260. The fluid-collection ring 270 can be configured such that when the shaft 200 is oriented horizontally (or approximately horizontally such as within about 15 degrees of a horizontal plane), the flared portion 1100 is located below the shaft 200 so as to be in position to direct fluids downward and away from the handle 260 during a medical procedure.

Other than the shape of the fluid-collection ring 270 and the flared portion 1100, the fluid-collecting ring 270 illustrated in FIGS. 11A and 11B can be the same as or combined with the fluid-collection ring 270 shown in FIGS. 2, 3A, 3B, 3C, 4, 5A, 5B, 6A, 6B, 7A, 7B, 8A, 8B, 9A, 9B, 10A, and/or 10B.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A medical device comprising:
an ultrasound applicator comprising:
a shaft having proximal and distal ends;
a plurality of channels defined in the shaft and extending from the proximal end of the shaft, including:
an ultrasound channel, and
a cooling channel;
one or more ultrasound transducers disposed in the ultrasound channel;
an ultrasound window defined in the shaft and aligned with the one or more ultrasound transducers; and
a handle attached to the proximal end of the shaft; and
a fluid-collection ring disposed on a distal side of the handle, the fluid-collection ring having an inner edge and an outer edge, the inner edge defining a hole through which the shaft extends, a distal side of the fluid-collection ring having a concave shape and configured to direct fluid away from the handle.

2. The medical device of claim 1, wherein the distal side of the fluid-collection ring is configured to conform to an entry point of a mammalian body.

3. The medical device of claim 2, wherein the distal side of the fluid-collection ring is configured to conform to a tip of a penis.

4. The medical device of claim 1, wherein a proximal side of the fluid-collection ring is attached to the distal side of the handle such that a rotational orientation of the fluid-collection ring remains stationary when the shaft is rotated.

5. The medical device of claim 1, wherein the inner edge and the shaft form a fluid-tight seal.

6. The medical device of claim 1, wherein the inner edge and the shaft define a fluid-collection channel and the medical device further comprises a fluid-collection reservoir that is fluidly coupled to the fluid-collection channel.

7. The medical device of claim 6, wherein the fluid-collection reservoir is disposed in the handle.

8. The medical device of claim 1, further comprising complementary keyed projections disposed on a proximal side of the fluid-collection ring and the distal side of the handle, the complementary keyed projections configured to mechanically engage one another to limit a rotation of the fluid-collection ring with respect to the shaft.

9. The medical device of claim 1, wherein the fluid-collection ring comprises a biocompatible polymer or a biocompatible plastic.

10. The medical device of claim 1, wherein a drainage hole is defined through a proximal side and the distal side of the fluid-collection ring and the medical device further comprises a drainage tube fluidly coupled to the drainage hole on the proximal side of the fluid-collection ring.

11. The medical device of claim 1, wherein the fluid-collection ring is elongated with respect to a first axis compared to with respect to a second axis, the shaft extending along a third axis, the first, second, and third axes mutually orthogonal.

12. A medical device comprising:
an ultrasound applicator comprising:
a shaft having proximal and distal ends;
a plurality of channels defined in the shaft and extending from the proximal end of the shaft, including:
an ultrasound channel, and
a cooling channel;
one or more ultrasound transducers disposed in the ultrasound channel;
an ultrasound window defined in the shaft and aligned with the one or more ultrasound transducers; and
a handle attached to the proximal end of the shaft; and
a fluid-collection ring disposed on a distal side of the handle, the fluid-collection ring having an inner edge, an outer edge, and a fluid-collection channel defined on a distal side of the fluid-collection ring.

13. The medical device of claim 12, wherein the fluid-collection channel is defined only on a distal surface of the fluid-collection ring.

14. The medical device of claim 13, wherein the fluid-collection channel extends to a hole defined in the outer edge of the fluid-collection ring.

15. The medical device of claim 14, wherein the hole is oriented at a bottom of the fluid-collection channel when the shaft is oriented parallel to a horizontal axis.

16. A medical apparatus comprising:
an ultrasound applicator comprising:
a shaft having proximal and distal ends;
a plurality of channels defined in the shaft and extending from the proximal end of the shaft, including:
an ultrasound channel, and
a cooling channel;
one or more ultrasound transducers disposed in the ultrasound channel;
an ultrasound window defined in the shaft and aligned with the one or more ultrasound transducers; and
a handle attached to the proximal end of the shaft;
a fluid-collection ring disposed on a distal side of the handle, the fluid-collection ring having an inner edge and an outer edge, the inner edge defining a hole through which the shaft extends, a distal side of the fluid-collection ring having a concave shape and configured to direct fluid away from the handle;
a power supply electrically coupled to the one or more ultrasound transducers;
a reservoir configured to store a cooling fluid, the reservoir fluidly coupled to the cooling channel; and
a pump that circulates the cooling fluid between the reservoir and the cooling channel.
